(19) United States
(12) Patent Application Publication
(10) Pub. No.: US 2019/0183354 A1
(43) Pub. Date: Jun. 20, 2019

(54) MONITORING BLOOD PRESSURE IN THE INFERIOR VENA CAVA

(71) Applicant: Edwards Lifesciences Corporation, Irvine, CA (US)

(72) Inventor: Stanton J. Rowe, Newport Coast, CA (US)

(21) Appl. No.: 16/215,872

(22) Filed: Dec. 11, 2018

Related U.S. Application Data
(60) Provisional application No. 62/607,121, filed on Dec. 18, 2017.

(51) Int. Cl. A61B 5/0215; A61B 5/07; A61B 5/00

(52) U.S. Cl. CPC A61B 5/0215; A61B 5/076; A61B 5/7275; A61B 5/746; A61B 2562/162; A61B 5/6882; A61B 5/741; A61B 5/486; A61B 2562/0247; A61B 5/6862

(57) ABSTRACT

Disclosed herein are devices, systems, and methods for monitoring blood pressure in the inferior vena cava (IVC) to diagnose acute decompensated heart failure (ADHF). An underlying principle of the disclosed systems and methods is that it is possible to reduce or prevent hospitalization by monitoring blood pressure in the IVC because an increase in IVC pressure is correlated with worsening renal function, causing fluid overload. Accordingly, the disclosed systems and methods monitor renal function by monitoring IVC pressure to detect early signs of ADHF by identifying increases in pressure over time. To monitor pressure in the IVC, a pressure sensor can be implanted in the IVC and can wirelessly transmit sensor data to a monitor device worn by the subject. If a pressure trend exceeds a specified threshold, the monitor device can generate an alert.

### CROSS-REFERENCE TO RELATED APPLICATIONS

The present application claims the benefit of priority to U.S. Provisional Application No. 62/607,121, filed Dec. 18, 2017, and entitled “MONITORING BLOOD PRESSURE IN THE INFERIOR VENA CAVA,” which is incorporated by reference in its entirety for all purposes.

### BACKGROUND

### Field

The present disclosure relates generally to monitoring blood pressure in the inferior vena cava, and more particularly to systems, apparatuses, and methods for measuring blood pressure in the inferior vena cava to diagnose fluid overload associated with acute congestive heart failure.

### Description of Related Art

Acute decompensated heart failure (ADHF) can be defined as a sudden or gradual onset of signs or symptoms of heart failure. In some instances, ADHF is a worsening of chronic heart failure symptoms that can result in acute respiratory distress. Where there is a rapid onset of symptoms and signs of heart failure, ADHF may also be referred to as acute congestive heart failure which may occur with or without previous cardiac disease. ADHF is the most common cause of hospitalization in patients 65 years and older. Rates of rehospitalization or death approach 50% within 6 months of patients experiencing ADHF.

ADHF nearly always includes pulmonary and systemic congestion. Because patients experiencing ADHF usually display volume overload, it may be beneficial to remove fluid to relieve the symptoms of heart failure and to improve oxygenation. A common approach is diuretic therapy (e.g., administration of loop diuretics) to reduce fluid overload. Diuresis generally lowers central venous and pulmonary capillary wedge pressures, which decreases pulmonary edema and often results in augmented forward-stroke volume and cardiac output. However, uncertainty remains about the safety and efficacy of various doses as well as means of administration. For example, a patient may not improve with the administration of lower doses of diuretics resulting in the administration of increased dosages even though higher doses are generally associated with poorer outcomes for patients experiencing ADHF.

### SUMMARY

In a first aspect, the present disclosure relates to a system for monitoring pressure trends in an inferior vena cava of a subject. The system includes a pressure measurement device implanted in the inferior vena cava of the subject, the pressure measurement device including a pressure sensor and a transceiver, the pressure measurement device configured to acquire pressure measurements and to wirelessly transmit the acquired pressure measurements. The system also includes a pressure monitor device configured to be worn by the subject externally, the pressure monitor device including a transceiver and system electronics, the pressure monitor device configured to wirelessly receive pressure measurements from the pressure measurement device, to determine a trend in the acquired pressure measurements, and to automatically generate an alert responsive to the determined trend exceeding a predetermined threshold.

In some embodiments of the first aspect, the predetermined threshold is an increase of at least 50% over a period of 24 hours.

In some embodiments of the first aspect, the pressure measurement device includes a stent to which the pressure sensor and the transceiver are secured. In further embodiments, the stent is configured to expand to a diameter of at least 15 mm and less than or equal to 35 mm In further embodiments, the stent includes material that is configured to change diameter after implantation, wherein the diameter is configured to change between 20 mm and 35 mm In further embodiments, the stent includes one or more anchors for securing the stent within the inferior vena cava. In further embodiments, a circumferential force applied by the stent is sufficient to secure the stent within the inferior vena cava. In yet further embodiments, the stent comprises a self-expanding nickel titanium alloy.

In some embodiments of the first aspect, the pressure sensor comprises an encapsulated pressure transducer. In some embodiments of the first aspect, the transceiver is configured to transmit radio frequency signals to the pressure monitor device. In some embodiments of the first aspect, the pressure monitor device is implemented in a belt. In some embodiments of the first aspect, the pressure monitor device is implemented in a watch.

In a second aspect, the present disclosure relates to a kit for a system for monitoring pressure trends in an inferior vena cava of a subject. The kit includes packaging configured to secure a pressure measurement device implanted in the inferior vena cava of the subject, the pressure measurement device including a pressure sensor and a transceiver, the pressure measurement device configured to acquire pressure measurements and to wirelessly transmit the acquired pressure measurements. The kit also includes packaging configured to secure a pressure monitor device configured to be worn by the subject externally, the pressure monitor device including a transceiver and system electronics, the pressure monitor device configured to wirelessly receive pressure measurements from the pressure measurement device, to determine a trend in the acquired pressure measurements, and to automatically generate an alert responsive to the determined trend exceeding a predetermined threshold.

In a third aspect, the present disclosure relates to a method for monitoring pressure in an inferior vena cava of a subject to diagnose early signs of acute decompensated heart failure. The method includes intermittently measuring blood pressure in the inferior vena cava. The method also includes determining, using a processor, a trend in blood pressure in the inferior vena cava based on the intermittent blood pressure measurements. The method also includes determining, using a processor, that the determined trend satisfies alert criteria wherein the alert criteria includes an increase in pressure of at least 50% in a 24-hour window. The method also includes indicating a diagnosis of fluid overload responsive to the determination that the determined trend satisfies the alert criteria.

In some embodiments of the third aspect, intermittently measuring blood pressure comprises acquiring blood pressure measurements using a pressure sensor at least two times in a 24-hour period. In some embodiments of the third aspect, determining the trend in blood pressure includes calculating a difference in pressure measurements over a pre-determined period of time. In further embodiments, the pre-determined period of time is 24 hours.

In some embodiments of the third aspect, determining the trend in blood pressure includes calculating a slope of a fit to acquired pressure measurements. In some embodiments of the third aspect, indicating the diagnosis of fluid overload comprises generating a visual alert. In some embodiments of the third aspect, indicating the diagnosis of fluid overload comprises generating an audible alert.

In some embodiments of the third aspect, the method further includes displaying a recommendation to increase intake of oral diuretics responsive to the determination that the determined trend satisfies the alert criteria. In some embodiments of the third aspect, the method further includes playing an audible message that recommends increasing intake of oral diuretics responsive to the determination that the determined trend satisfies the alert criteria.

For purposes of summarizing the disclosure, certain aspects, advantages and novel features have been described herein. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 illustrates an example embodiment of a blood pressure monitoring system with a blood pressure measurement device for implantation in the inferior vena cava.

FIGS. 2A, 2B, and 2C illustrate an example of implanting a blood pressure measurement device in a subject to monitor blood pressure in the inferior vena cava.

FIG. 3 illustrates a block diagram of a blood pressure monitoring system having a pressure monitor and a pressure measurement system for implantation in the inferior vena cava.

FIG. 4 illustrates a flow chart of an example method for measuring blood pressure in the inferior vena cava to diagnose fluid overload associated with acute congestive heart failure.

FIG. 5 illustrates a flow chart of an example method for determining blood pressure trends in the inferior vena cava to diagnose fluid overload associated with acute congestive heart failure.

FIG. 6 illustrates a flow chart of an example method for monitoring blood pressure in the inferior vena cava to diagnose fluid overload associated with acute congestive heart failure.

FIG. 7 illustrates a flow chart of an example method for implanting a blood pressure measurement device for monitoring pressure in the inferior vena cava.

### DETAILED DESCRIPTION

The headings provided herein are for convenience only and do not necessarily affect the scope or meaning of any of the claimed embodiments.

### Overview

There has been relatively little development of new therapies in acute decompensated heart failure (ADHF) over several decades. Typically, loop diuretics are a mainstay of therapy for patients in ADHF. The underlying goal of this therapy is to reduce fluid or volume overload to improve heart function. This is because patients in ADHF are hospitalized due to fluid or volume overload rather than a failure of the heart to perform its core functions. Relatedly, cardiorenal syndrome, cardiogenic shock, and ADHF are related conditions that refer to the inability of a subject to maintain fluid balance as a result of acute or chronic dysfunction of one or more organs. For typical patients experiencing fluid overload (e.g., due to cardiorenal syndrome, cardiogenic shock, ADHF, etc.), congestion and fluid overload may be caused by worsening renal function. Unfortunately, the safety and efficacy of various doses of diuretics, as well as the means of administration, are not fully understood. It would be advantageous, then, to improve the diagnosis and resulting treatment of patients that may experience ADHF by focusing on indicators of renal function.

Accordingly, described herein are devices, systems, and methods for monitoring blood pressure in the inferior vena cava (IVC) to diagnose ADHF, acute congestive heart failure, and other similar conditions. An underlying principle of the disclosed systems and methods is the realization that it is possible to reduce or prevent hospitalization by monitoring blood pressure in the IVC because an increase in IVC pressure is correlated with worsening renal function, causing fluid overload. Accordingly, the disclosed systems and methods monitor renal function by monitoring IVC pressure. This is done to detect early signs of ADHF or acute congestive heart failure by identifying increases in pressure over time.

The correlation between IVC pressure and renal function is due at least in part to renal venous congestion being a strong hemodynamic determinant for worsening renal function. This relationship may be understood by comparing parameters of the venous system and the arterial system. Where the heart is failing to perform normally, cardiac output may be limited which in turn causes worsening renal function. High central venous pressure occurs because the renal venous pressure is high and the glomerular filtration rate (GFR) drops. In turn, high central venous pressure causes deteriorating renal function. In addition, it is typical to observe a high atrial pressure that may cause an approximately three-fold drop in filtration pressure. This combination of parameters indicates worsening renal function with a key indicator being a high central venous pressure. Accordingly, monitoring pressure in the IVC can be advantageous in diagnosing early signs of ADHF or acute congestive heart failure because it is an indicator of renal function.

Monitoring pressure in the IVC is preferable to monitoring pulmonary pressure and/or filtration pressure because a key issue in ADHF is fluid overload. ADHF is not generally caused by changes in the heart, but rather by worsening renal function leading to fluid overload. To monitor renal function, it is more efficacious to measure IVC pressure than to measure pulmonary pressure. This is due at least in part to pulmonary pressures being indicators of heart function. However, for patients in ADHF, it is not the heart function that is the underlying cause, but rather kidney function. Consequently, monitoring IVC pressure is superior in reducing or preventing hospitalization for patients in ADHF experiencing fluid overload because IVC pressure is correlated with fluid volume overload, as described herein.

There are additional advantages to monitoring pressure in the IVC as compared to monitoring pulmonary pressure and/or filtration pressure. For example, it is relatively easy to implant a pressure sensor in the IVC without the use of x-ray imaging or without using any type of imaging (e.g., fluoroscopy). As another example, a pressure sensor can be implanted in the IVC without requiring the patient first visit a catheterization laboratory. As another example, the process of implanting a pressure sensor in the IVC is forgiving because the IVC is a relatively large venous vessel. Implantation generally involves guiding a catheter through the femoral vein and delivering the pressure sensor to the IVC just above the bifurcation. Furthermore, increases in pressure in the IVC indicate early signs of acute congestive heart failure. Relative to a pulmonary pressure sensor, there are fewer challenges to implanting an IVC pressure sensor and to acquiring and transmitting measurements to a monitoring device outside of a patient's body. A pulmonary pressure sensor contends with the lungs, chest, and heart when trying to communicate measurements to an external monitoring system whereas there are no such challenges for an IVC pressure monitor to transmit data to an external monitoring system positioned just above the pelvic bone. Advantageously, this allows the external monitoring system to be something that can be worn by a patient (e.g., as part of a belt or a watch).

The described systems and methods enable automated diagnosis of ADHF and/or early indication of ADHF based at least in part on the trend of blood pressure measurements in the IVC. For example, where blood pressure measurements in the IVC increase over time, the disclosed systems and methods may generate an indicator or alert of potential ADHF. As a result, an increase in oral diuretics may be prescribed or recommended to reduce or to prevent instances of hospitalization in patients. In some embodiments, an increase of at least about 50% in IVC pressure over a period of about 24 hours can cause the disclosed systems and methods to diagnose early signs of ADHF. In certain embodiments, the systems and methods are configured to generate an indicator or an alert where, over a pre-determined time, an IVC pressure sensor measures an increase in pressure of at least about 25%, at least about 40%, at least about 50%, at least about 60%, at least about 75%, or at least about. In various implementations, the pre-determined period of time can be at least about 12 hours and/or less than or equal to about 96 hours, at least about 18 hours and/or less than or equal to about 90 hours, at least about 24 hours and/or less than or equal to about 84 hours, at least about 36 hours and/or less than or equal to about 78 hours, at least about 48 hours, or at least about 72 hours. It is preferable to use a percentage change of IVC pressure in determining early signs of ADHF rather than an absolute value of IVC pressure due at least in part to subjects with heart failure or related conditions typically having abnormal IVC pressures. Thus, these subjects have a wide range of IVC pressures that may be outside of typical or average values. However, it is to be understood that an absolute value or threshold of IVC pressure may be used in certain implementations.

### Example IVC Pressure Monitoring Systems

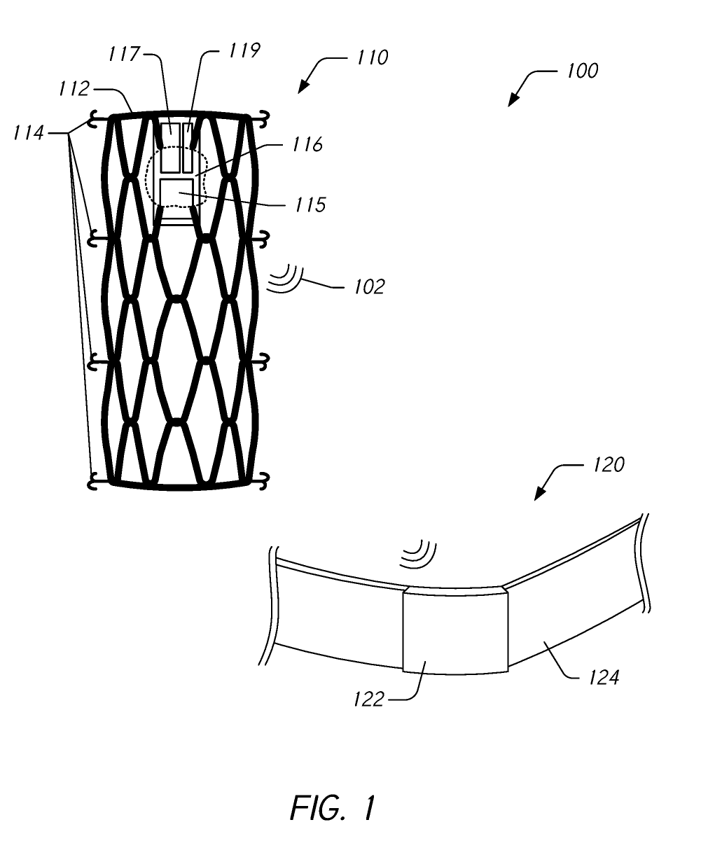

FIG. 1 illustrates an example embodiment of a blood pressure monitoring system 100 with a pressure measurement device 110 for implantation in the IVC. The system 100 also includes a pressure monitoring device 120 for use outside of a subject's body. The pressure measurement device 110 is configured to acquire pressure measurements and to wirelessly transmit signals 102 indicative of the measurements to the pressure monitoring device 120. The pressure monitoring device 120 is configured to receive the wirelessly transmitted signals 102 and to track trends in IVC pressure over time. If the trend indicates early signs of ADHF (e.g., increasing IVC pressure), as described herein, the pressure monitoring device 120 is configured to generate an indicator or alert to a subject or to a clinician. Accordingly, the system 100 is configured to provide an automated diagnosis of ADHF based at least in part on trends in IVC pressures, which can indicate worsening renal function. This automated diagnosis can reduce or prevent hospitalizations by prompting a subject to take action to reduce fluid volume overload prior to requiring hospitalization. For example, the system 100 can recommend an increase in oral diuretics to decrease fluid volume overload.

The pressure measurement device 110 can include a stent 112 configured to secure a sensor module 116 within the inferior vena cava of a subject. The stent 112 can include one or more anchors 114 or other securing means that are configured to secure the stent 112 within the IVC. The stent 112 can be made of a material that allows the stent 112 to be delivered in a compact state and to expand to a deployed state that secures it within the IVC. After deployment, the stent 112 is configured to expand and to contract to match the changing inner diameter of the IVC walls where it is implanted. In addition, the stent 112 can be configured to apply a relatively low radial force to be compatible with the wall tension of the IVC (e.g., where the low radial force is compatible with the relatively low wall tension of the IVC as compared to artery walls). For example, the radial force applied by the stent 112 can be less than the radial force applied by a typical stent designed for implantation in artery walls. This is advantageous because if the stent 112 were to apply a radial force comparable to a stent designed for arterial implantation, damage may be done to the walls of the IVC. In some embodiments, the pressure measurement device 110 is configured to be permanently implanted in the subject. In some embodiments, the stent 112 can expand to have a diameter that is at least about 10 mm and/or less than or equal to about 45 mm, at least about 15 mm and/or less than or equal to about 40 mm, or at least about 20 mm and/or less than or equal to about 35 mm In certain implementations, the diameter of the stent 112 after implantation in the IVC can vary between about 20 mm and about 35 mm to accommodate changes in the inner diameter of the IVC.

In some embodiments, the stent 112 may be crimped or otherwise mounted on an intravascular balloon catheter and delivered to the IVC. This balloon catheter can then be inflated, forcing the anchors 114 of the stent 112 to contact the vessel wall to secure the stent 112 to the vessel wall. In some embodiments, the sensor module 116 can be folded within a self-expanding stent 112 constructed from a thermal memory metal such as nitinol. The nitinol stent 112 can be introduced into the IVC and allowed to expand using standard techniques. As the stent 112 expands, the sensor module 116 unfolds into its targeted final (e.g., flat) shape. The stent 112, which is held fixed against the wall of the IVC due to the self-expanding nature of the nitinol materials exerting an outward radial force, may serve as the mechanism to keep the sensor module 116 fixed in a targeted position within the vasculature. In such embodiments, the anchors 114 may or may not be part of the stent 112.

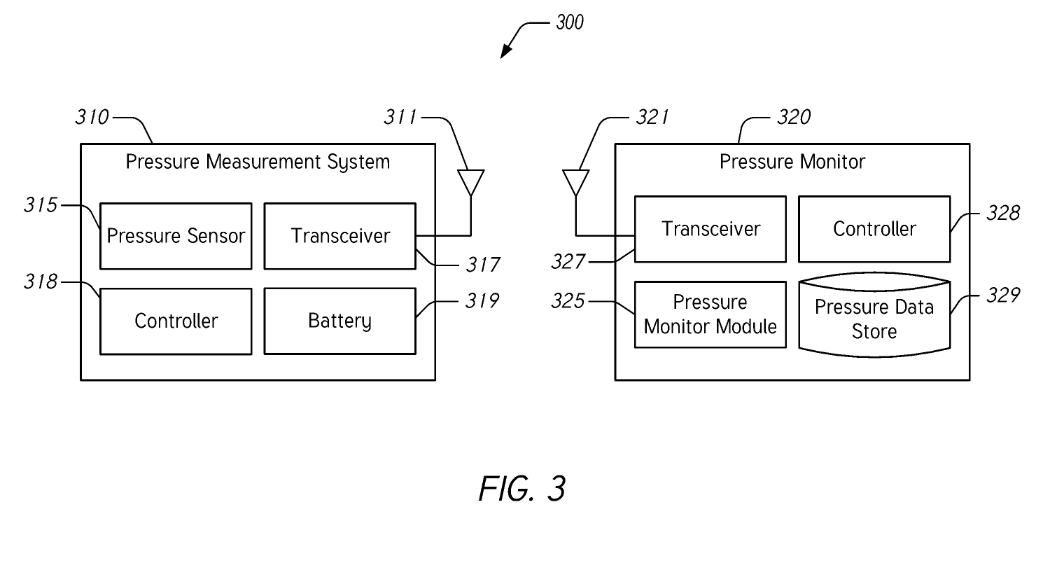

The sensor module 116 includes a pressure sensor 115, a transceiver 117, and a battery 119 or other power source. As described in greater detail herein with reference to FIG. 3, the pressure sensor 115 is configured to acquire pressure measurements within the IVC. The measurements are converted into signals by the pressure sensor 115 and/or the transceiver 117 and the transceiver wirelessly transmits signals 102 using an antenna. The battery 119 is electrically coupled and provides electrical power to the pressure sensor 115 and/or to the transceiver 117.

The pressure sensor 115 can be a pressure transducer. The pressure sensor 115 can be encapsulated and isolated from the blood. In some embodiments, the pressure sensor 115 can be manufactured using micro-machining techniques that were developed for the integrated circuit industry. An example of this type of sensor features an inductive-capacitive (LC) resonant circuit with a variable capacitor, examples of which are described in Allen et al., U.S. Pat. No. 6,111,520, entitled “System and method for the wireless sensing of physical properties,” the entirety of which is incorporated herein by reference. Such a sensor can contain two types of passive electrical components: an inductor and a capacitor. This type of pressure sensor can be constructed so that the fluid pressure at the sensor's surface changes the distance between the capacitor's parallel plates and causes a variation of the sensor's capacitance. Further description and examples of suitable pressure sensors is provided in Allen at al., U.S. Pat. No. 7,147,604, entitled “High Q factor sensor,” the entirety of which is incorporated herein by reference.

The transceiver 117 can be configured to generate radio-frequency (RF) signals for communication with the pressure monitoring device 120. The transceiver 117 can be configured to generate ultrasound signals for communication with the pressure monitoring device 120. In some embodiments, the transceiver 117 is configured to receive signals from the pressure monitoring device 120 or other compatible device. These received signals can be used to control operation of the sensor module 116, e.g., to initiate a pressure measurement, to initiate transmission of pressure measurement data, and the like.

The pressure monitoring device 120 can be implemented as a wearable device. The pressure monitoring device 120 can include a wireless transceiver configured to receive the wireless signals 102 from the pressure measurement device 110. The pressure monitoring device 120 includes an electronic housing 122 and a wearable element 124. The electronic housing 122 houses system electronics configured to receive wireless signals, to provide power to electronic components, to analyze the received signals, to store data, to generate indicators or alerts, to display information, and/or to generate sound, light, images, animations, and/or videos. The wearable element 124 can be a strap or other similar component configured to secure the electronic housing 122 to a subject or to an article of clothing. For example, the pressure monitoring device 120 can be implemented as a belt so that the electronic housing 122 is positioned near the bifurcation of the IVC to receive the wireless signals 102. As another example, the pressure monitoring device 120 can be implemented as a watch or part of a watch. In certain implementations, the functionality of the pressure monitoring device 120 is incorporated into an electronic device such as a smartphone, tablet, or smart watch.

In some embodiments, the pressure monitoring system 100 can be included in a kit. The kit can include the pressure measurement device 110 and the pressure monitoring device 120 as well as packaging for the devices 110, 120.

### Example Implantation of IVC Pressure Monitoring Systems

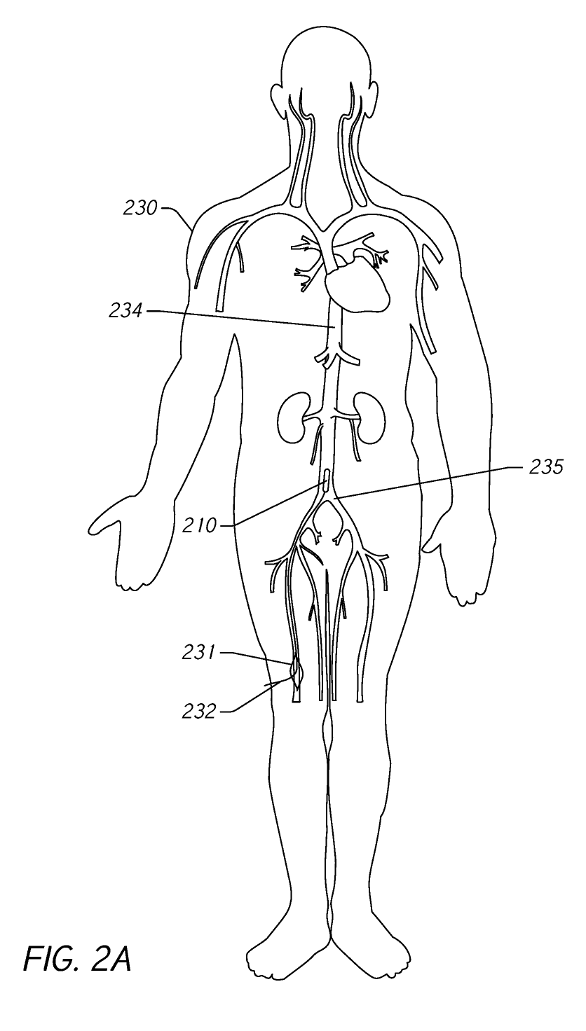

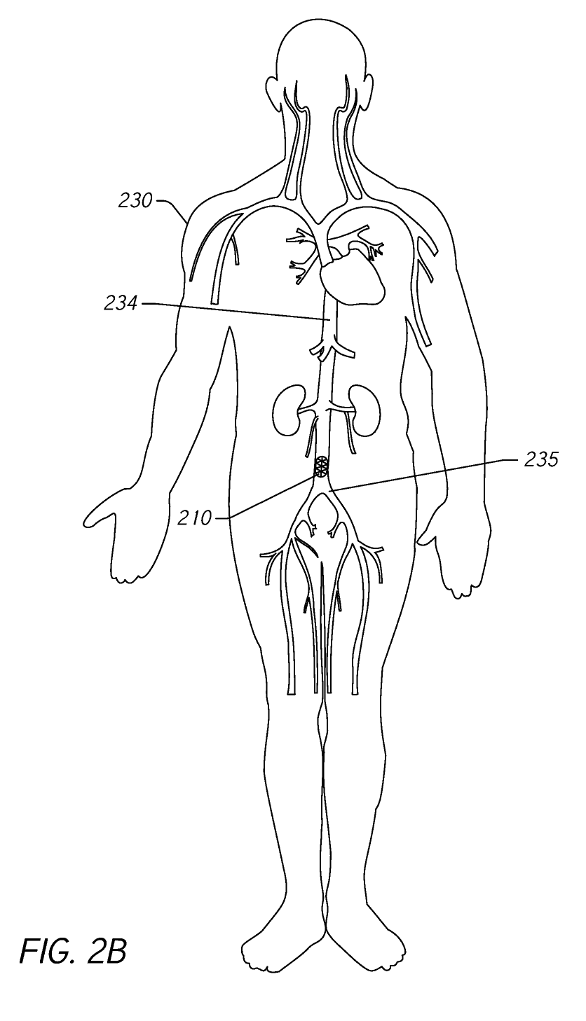

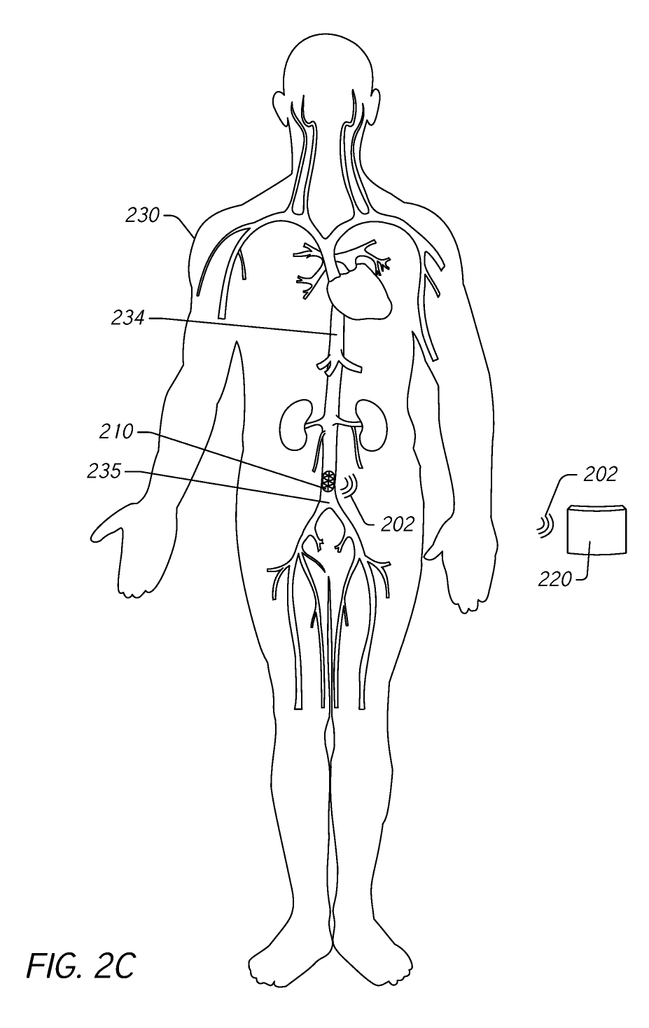

FIGS. 2A, 2B, and 2C illustrate an example of implanting a blood pressure measurement device 210 in a subject 230 to monitor blood pressure in the IVC 234. The blood pressure measurement device 210 is similar to the pressure measurement device 110 described herein with reference to FIG. 1 and includes the same or similar functionality and structure. The subject 230 is illustrated with a representation of a portion of the vasculature system to illustrate the inferior vena cava 234 within the subject 230. However, it is to be understood that no dimensions or relative sizes of components may be inferred from the relative sizes and dimensions of elements in the figures.

FIG. 2A illustrates delivery of the pressure measurement device 210 using a catheter or other vasculature access device. Access is gained to a femoral vein 231 through an incision 232 in the leg of the subject 230. The pressure measurement device 210 is advanced through the femoral vein 231 to the IVC 234 just above the bifurcation 235 of the IVC 234. As described herein, this may be accomplished without the aid of x-ray imaging or other types of imaging during delivery and implantation. In some embodiments, implantation may be accomplished without the use of a catheterization lab. In some embodiments, implantation may be accomplished using portable fluoroscopy. Due at least in part to the relative ease of implantation, this procedure may reduce costs, reduce the use of resources, and increase convenience when compared to implanting a pressure monitor near the heart (e.g., the pulmonary artery).

FIG. 2B illustrates implantation of the pressure measurement device 210 in the IVC 234. Once positioned above the bifurcation 235, the pressure measurement device 210 can be expanded within the IVC 234. As described herein, the pressure measurement device 210 can include a stent comprising self-expanding material such as a nickel titanium alloy (e.g., Nitinol) or the stent can be inflated using, for example, a balloon catheter. In its expanded form, the pressure measurement device 210 secures itself within the IVC 234. This can be accomplished, for example, using anchors or other securing means or the pressure measurement device 210 can secure itself within the IVC 234 due to radial forces applied by the pressure measurement device 210.

FIG. 2C illustrates the pressure monitoring system 200 functioning to monitor pressure in the IVC 234. The pressure measurement device 210 transmits wireless signals 202 to a pressure monitoring device 220. The pressure monitoring device 220 receives the wireless signals and determines trends in the IVC pressure measured by the pressure measurement device 210. If the determined trend exceeds a threshold, the pressure monitoring device 220 can generate an indicator or alert. Responsive to the indicator or alert, the subject 230 can take suitable action such as increasing intake of oral diuretics.

Accordingly, the pressure monitoring system 200 can be configured to acquire pressure measurements within the IVC 234 of the subject 230 and to transmit the measurements to an external pressure monitoring device 220 that tracks trends in IVC pressure measurements. The pressure monitoring system 200 is configured to provide an automated diagnosis of early signs of ADHF.

By implanting the pressure measurement device 210 in the IVC 234 near the bifurcation 235, the pressure measurement device 210 is relatively free from internal obstructions that may impede connectivity between the pressure measurement device 210 and the pressure monitoring device 220. For the sake of comparison, a pulmonary artery pressure sensor is forced to contend with the lungs, chest, and heart in establishing wireless or wired connectivity with a pressure monitoring device. This is more complex than a site just above the pelvic bone, as is the case for the disclosed pressure measurement device 210. In addition, the pressure measurement device 210 can be positioned near the pressure monitoring device 220 when it is implemented as a wearable device to increase connectivity and/or to reduce the likelihood of failed wireless communications between the pressure measurement device 210 and the pressure monitoring device 220.

### Example IVC Pressure Monitoring System Block Diagram

FIG. 3 illustrates a block diagram of a blood pressure monitoring system 300 having a pressure measurement system 310 for implantation in the IVC of a subject and a pressure monitor 320. The pressure monitoring system 300 is configured to provide an automated diagnosis of early signs of ADHF, as described herein.

The pressure measurement system 310 can include hardware, software, and/or firmware components used to control a pressure sensor 315 and to transmit pressure measurements using the transceiver 317. In some embodiments, the pressure measurement system 310 can be configured to receive wireless signals from the pressure monitor 320 to control operation of the pressure measurement system 310 (e.g., to initiate pressure measurements, to transmit pressure measurement data, and the like). The pressure measurement system 310 includes a pressure sensor 315, a transceiver 317, a controller 318, and a battery 319. Components of the pressure measurement system 310 can communicate with one another and with the pressure monitor 320. The pressure sensor 315 of the pressure measurement system 310 can be any suitable pressure sensor for determining blood pressure in the IVC, examples of which are described herein. The transceiver 317 is coupled to an antenna 311 and can be configured to transmit radio frequency signals, ultrasonic signals, or other wireless signals to communicate pressure measurements acquired by the pressure sensor 315. The controller 318 can be configured to control operation of the pressure sensor 315 and the transceiver 317. The controller 318 can include any suitable microprocessor and other computing components configured to interface with the pressure sensor 315 and transceiver 317. The battery 319 is electrically coupled to the controller 318, to the pressure sensor 315, and to the transceiver 317 to provide electrical power to these components.

As described herein, the pressure monitor 320 can be embodied in a wearable or other electronic device associated with (e.g., carried or worn by) the subject. The pressure monitor 320 can include hardware, software, and/or firmware components used to control a pressure monitor module 325, to receive pressure measurements using the transceiver 327, to analyze received pressure measurements to determine trends, and to generate indicators or alerts where the determined trends exceed predetermined thresholds. In some embodiments, the pressure monitor 320 can be configured to transmit wireless signals to the pressure measurement system 310 to control its operation (e.g., to initiate pressure measurements, to transmit pressure measurement data, and the like). The pressure monitor 320 includes a pressure monitor module 325, a transceiver 327, a controller 328, and a pressure data store 329. Components of the pressure monitor 320 can communicate with one another and with the pressure measurement system 310. The transceiver 327 is coupled to an antenna 321 and can be configured to receive and/or to transmit radio frequency signals, ultrasonic signals, or other wireless signals to receive pressure measurements acquired by the pressure measurement system 310 and/or to interact with the pressure measurement system 310.

The pressure monitor module 325 can include any suitable combination of hardware, software and/or firmware to provide analysis of pressure measurement data. The pressure monitor module 325 can interface with the controller 328 to control operation of the module 325 and with the pressure data store 329 to store and to retrieve pressure measurement and other related data. The pressure monitor module 325 is configured to receive pressure measurement data and to determine trends over time of measured pressures in the IVC. In addition, the pressure monitor module 325 is configured to determine whether the determined trends exceed a predetermined threshold. For example, if the measured pressure has increased by at least about 50% during a 24-hour window, the pressure monitor module 325 can generate an alert or an indication that acts as an automated diagnosis of early signs of ADHF. The predetermined threshold can be based at least in part on different time windows. For example, the time window can be a 12-hour window, an 18-hour window, a 24-hour window, a 36-hour window, a 48-hour window, a 72-hour window, etc. Furthermore, the predetermined threshold can be based at least in part on different trends in pressure measurement. For example, the trend in the pressure measurement can be determined to exceed the predetermined threshold where the trend over the specified time window exceeds at least a 25% increase, at least a 35% increase, at least a 45% increase, at least a 50% increase, at least a 60% increase, at least a 75% increase, or at least a 100% increase. Where pressure trends exceed the predetermined threshold within the designated time window, the pressure monitor module 325 is configured to provide an automated diagnosis of early signs of ADHF.

The controller 328 can be configured to control operation of the pressure monitor module 325, the transceiver 327, and the pressure data store 329. The controller 328 can include any suitable microprocessor and other computing components configured to interface with the pressure monitor module 325 and the transceiver 327.

The pressure data store 329 is configured to store pressure measurements, calibration constants, biographical information, algorithms, executable instructions (e.g., instructions for the pressure monitor module 325 and/or the controller 328), and the like. The pressure data store 329 can be any suitable data storage device or combination of devices including, for example and without limitation, random access memory, read-only memory, solid-state disks, hard drives, flash drives, bubble memory, and the like.

### Methods for Diagnosing ADHF in a Subject

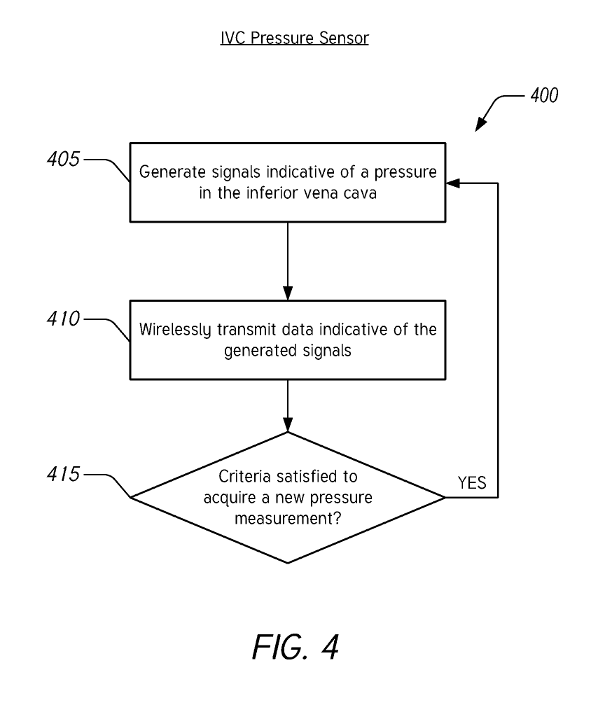

FIG. 4 illustrates a flow chart of an example method 400 for measuring blood pressure in the IVC to diagnose fluid overload associated with acute congestive heart failure. The method 400 can be performed by a pressure measurement system such as the pressure measurement device 110 described herein with reference to FIG. 1, the pressure measurement system 210 described herein with reference to FIG. 2, or the pressure measurement system 310 described herein with reference to FIG. 3, or any suitable component of these systems. For ease of description, the method 400 will be described as being performed by a pressure measurement system, but it should be understood that any step, any combination of steps, or any portion of a step of the method 400 can be performed by any suitable component of a pressure measurement system.

In block 405, the pressure measurement system generates signals indicative of a pressure in the inferior vena cava. The pressure measurement system can include one or more pressure sensors implanted in the IVC and configured to generate signals indicative of a pressure within the IVC. The pressure sensors can include transducers or other electrical components configured to generate electrical signals in response to pressures in the IVC. The one or more pressure sensors can be implanted and secured within the IVC using a stent or other suitable apparatus, as described herein.

In block 410, the pressure measurement system wirelessly transmits the data indicative of the generated signals. The pressure measurement system is configured to convert signals acquired by a pressure sensor into signals suitable for wireless transmission using, for example, a transceiver as described herein. The data transmitted wirelessly can be the raw data acquired by the pressure sensors or the data can be processed prior to transmission by any suitable component of the pressure measurement system, such as a controller or a transceiver as described herein.

In block 415, the pressure measurement system determines whether criteria have been satisfied to acquire a new pressure measurement. Responsive to this determination, the pressure measurement system returns to block 405 to acquire a new pressure measurement in the IVC. In some embodiments, the pressure measurement system is configured to acquire pressure measurements intermittently. In such embodiments, the criteria include an elapsed time since the last acquired and successfully transmitted measurement. In some embodiments, the criteria include whether a signal has been received from an external system to initiate a new pressure measurement. In such embodiments, the pressure measurement system can be configured to receive wireless communication wherein the wireless communication includes commands The commands can include, for example without limitation, instructions to initiate a new pressure measurement and/or to transmit one or more previous pressure measurements. In some embodiments, the criteria can be updated or changed based on previous measurements or based on received commands from an external system. In some embodiments, the pressure measurement system is configured to acquire pressure measurements in regular intervals. In certain embodiments, the pressure measurement system is configured to acquire pressure measurements in non-regular intervals. In various implementations, the pressure measurement system is configured to acquire pressure measurements at least one time daily, at least two times daily, at least three times daily, at least four times daily, or at least five times daily.

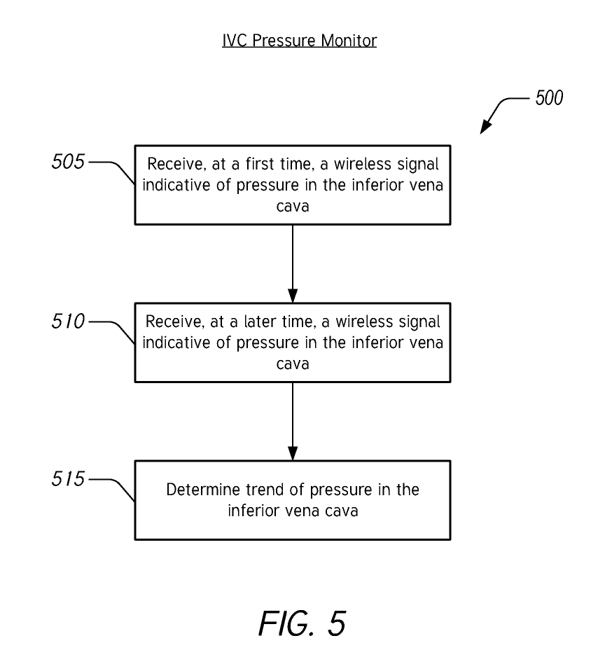

FIG. 5 illustrates a flow chart of an example method 500 for determining blood pressure trends in the IVC to diagnose fluid overload associated with acute congestive heart failure. The method 500 can be performed by a pressure monitor such as the pressure monitoring device 120 described herein with reference to FIG. 1, the pressure monitoring device 220 described herein with reference to FIG. 2, or the pressure monitor 320 described herein with reference to FIG. 3, or any suitable component of these devices. For ease of description, the method 500 will be described as being performed by a pressure monitor, but it should be understood that any step, any combination of steps, or any portion of a step of the method 500 can be performed by any suitable component of a pressure monitor.

In block 505, the pressure monitor is configured to receive, at a first time, a wireless signal indicative of pressure in the IVC. In block 510, the pressure monitor is configured to receive, at a later time, a wireless signal indicative of pressure in the IVC. In block 515, the pressure monitor is configured to determine a trend of pressure in the IVC. In some embodiments, the pressure monitor is configured to determine the trend by calculating a change in pressure divided by the time between the measured pressures. In certain embodiments, the pressure monitor is configured to determine the trend by calculating a derivative of a fit to previous pressure measurements. In various embodiments, the pressure monitor is configured to determine the trend using moving averages.

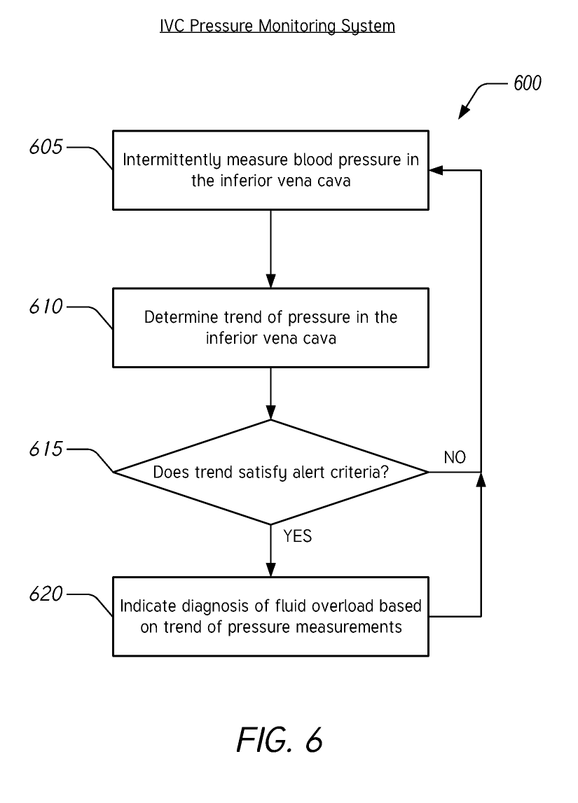

FIG. 6 illustrates a flow chart of an example method 600 for monitoring blood pressure in the IVC to diagnose fluid overload associated with acute congestive heart failure. The method 600 can be performed by a pressure monitoring system such as the pressure monitoring system 100 described herein with reference to FIG. 1, the pressure monitoring system 200 described herein with reference to FIG. 2, or the pressure monitoring system 300 described herein with reference to FIG. 3, or any suitable component of these systems. For ease of description, the method 600 will be described as being performed by a pressure monitoring system, but it should be understood that any step, any combination of steps, or any portion of a step of the method 600 can be performed by any suitable component of a pressure monitoring system.

In block 605, the pressure monitoring system intermittently measures blood pressure in the IVC. This can be accomplished, for example, by a pressure measurement device using the method 400 described herein with reference to FIG. 4.

In block 610, the pressure monitoring system determines a trend of pressure in the IVC. This can be accomplished, for example, by a pressure monitor using the method 500 described herein with reference to FIG. 5.

In block 615, the pressure monitoring system compares the determined trend to alert criteria. The alert criteria include a predetermined threshold for the trend determined in block 610. As described herein, the alert criteria can be satisfied where an increase in pressure in the IVC is greater than or equal to a specified amount within a specified time window. The specified amount can be, for example and without limitation, 25%, 35%, 45%, 50%, 60%, 75%, 100%, etc. The specified time window can be, for example and without limitation, 12 hours, 18 hours, 24 hours, 48 hours, 72 hours, etc.

Responsive to a negative result in the comparison, the pressure monitoring system returns to block 605 to acquire additional pressure measurements. Responsive to a positive result in the comparison, the pressure monitoring system moves to block 620 to indicate a diagnosis of fluid overload based on the trend of pressure measurements. The indication can be in the form of an alert provided by a monitor or other device. The alert or indication can be a visual display and/or an audible sound. This can be used as an automated diagnosis of early signs of ADHF, as described herein.

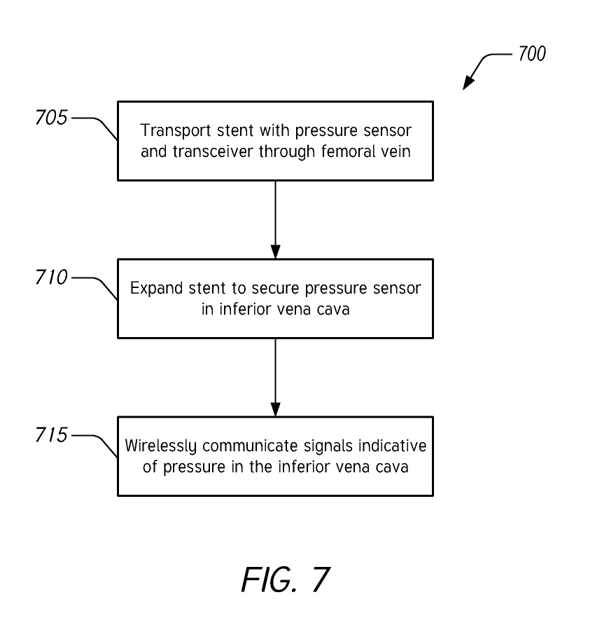

FIG. 7 illustrates a flow chart of an example method 700 for implanting a blood pressure measurement device for monitoring pressure in the IVC. The blood pressure measurement device can be any suitable pressure sensor or pressure measurement system such as the pressure measurement device 110 described herein with reference to FIG. 1, the pressure measurement system 210 described herein with reference to FIG. 2, or the pressure measurement system 310 described herein with reference to FIG. 3.

In block 705, a stent is transported through the femoral vein to the IVC. The stent can be transported using a vascular access device, such as a catheter. The stent includes a pressure sensor and a transceiver for acquiring and transmitting pressure measurements to an external system. In some embodiments, the stent can be transported to a desired or targeted location in the IVC without the help of imaging technologies.

In block 710, once the stent is positioned above the bifurcation in the IVC, the stent is expanded to secure the pressure sensor in place in the IVC. The stent can be expanded due to the use of self-expanding material (e.g., a nickel titanium alloy), using an expanding tool (e.g., a balloon catheter), or a combination of these techniques. Securing the stent in the IVC can include the stent applying a relatively low radial force on the IVC wall in comparison to a stent designed for implantation in an artery. In some embodiments, the stent includes one or more anchors to secure it to the vessel wall. In some embodiments, the stent is held in place due at least in part to a radial force applied by the stent in its deployed state. The stent can be configured to expand and contract after implantation to accommodate for changes in the inner diameter of the IVC.

In block 715, implantation of the stent can cause the pressure sensor to begin to acquire and to transmit pressure measurements. The transmitted pressure measurements can be received by an external system such as a pressure monitor system, as described herein. In this way, a pressure sensor in the IVC can acquire pressure measurements that are transmitted to an external system that determines pressure measurement trends to automatically diagnose early signs of ADHF, as described herein.

### Additional Embodiments and Terminology

The terms acute decompensated heart failure (ADHF) and acute congestive heart failure are used interchangeably herein and relate to a sudden or gradual onset of signs or symptoms of heart failure. Accordingly, the disclosed devices, systems, and methods can be used for the automated diagnosis of ADHF and/or acute congestive heart failure as well as other related conditions associated with fluid overload.

The terms “subject” and “patient” are used interchangeably herein and relate to mammals, inclusive of warm-blooded animals (domesticated and non-domesticated animals), and humans. The terms “clinician” and “healthcare provider” are used interchangeably herein.

The phrase “vascular access device” as used herein relates to any device that is in communication (or contact) with the vascular system of a subject. Vascular access devices include but are not limited to catheters, shunts, blood withdrawal devices, connectors, fluid couplers, valves, tubing and the like.

The term “sensor” as used herein relates to a device, component, or region of a device capable of detecting and/or quantifying and/or qualifying a physiological parameter of a subject. The phrase “system” as used herein relates to a device, or combination of devices operating at least in part in a cooperative manner, that is inclusive of the “sensor.” Sensors generally include those that continually measure the physiological parameter without user initiation and/or interaction (“continuous sensing device” or “continuous sensor”). Continuous sensors include devices and monitoring processes wherein data gaps can and/or do exist, for example, when a continuous pressure sensor is temporarily not providing data, monitoring, or detecting. Sensors also generally include those that intermittently measure the physiological parameter with or without user initiation and/or interaction (“intermittent sensing device” or “intermittent sensor”). In some embodiments, sensors, continuous sensing devices, and/or intermittent sensing devices relate to devices, components, or regions of devices capable of detecting and/or quantifying and/or qualifying a physiological hemodynamic parameter of a subject.

The phrases “physiological data,” “physiological parameter,” and/or “hemodynamic parameter” include without limitation, parameters directly or indirectly related to providing or calculating blood pressure (BP), IVC blood pressure, stroke volume (SV), cardiac output (CO), end-diastolic volume, ejection fraction, stroke volume variation (SVV), pulse pressure variation (PPV), systolic pressure variations (SPV), extravascular lung water index (ELWI), pulmonary vascular permeability index (PVPI), global end-diastolic index (GEDI), global ejection fraction (GEF), systolic volume index (SVI), arterial blood pressure (ABP), cardiac index (CI), systemic vascular resistance index (SVRI), peripheral resistance (PR), central venous saturation (ScvO2), and plethysmographic variability index (PVI). Hemodynamic parameters are inclusive of the absolute value of such parameters, a percentage change or variation in the parameters since an event was recorded, and an absolute percentage change within a previous time segment.

The phrases “electronic connection,” “electrical connection,” “electrical contact” as used herein relate to any connection between two electrical conductors known to those in the art. In some embodiments, electrodes are in electrical connection with (e.g., electrically connected to) the electronic circuitry of a device.

The term and phrase “electronics” and “system electronics” as used herein relate to electronics operatively coupled to the sensor and configured to measure, process, receive, and/or transmit data associated with a sensor, and/or electronics configured to communicate with a monitor or a data acquisition device.

The term “monitor” as used herein as a noun, refers to a device configured to observe, record, oversee, detect, supervise, regulate, receive, and/or transmit one or more signals, operations or conditions over a fixed, intermittent, or continuous period of time, for example, signals from a blood pressure sensor. The monitor can include a display for presenting data or other information. The monitor can include one or more processors or processing modules.

The term “display” as used herein as a noun, refers to a device configured to provide a visual representation of data (e.g., text and/or graphics and/or symbols) or any other information from a processor, computer, or monitor.

The term and phrase “controller,” “processor” or “processing module,” as used herein relates to components and the like designed to perform arithmetic or logic operations using logic circuitry that responds to and processes basic instructions, for example, instructions that drive a computer and/or perform calculations of numbers or their representation (e.g., binary numbers).

The terms “substantial” and “substantially” as used herein relate to a sufficient amount that provides a desired function. For example, an amount greater than 50 percent, an amount greater than 60 percent, an amount greater than 70 percent, an amount greater than 80 percent, or an amount greater than 90 percent.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the present specification supersedes and/or takes precedence over any such contradictory material of the incorporated reference.

Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described herein. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments. The terms “comprising,” “including,” “having,” “characterized by,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

Reference throughout this specification to “certain embodiments” or “an embodiment” means that a particular feature, structure or characteristic described in connection with the embodiment is included in at least some embodiments. Thus, appearances of the phrases “in some embodiments” or “in an embodiment” in various places throughout this specification are not necessarily all referring to the same embodiment and may refer to one or more of the same or different embodiments. Furthermore, the particular features, structures or characteristics can be combined in any suitable manner, as would be apparent to one of ordinary skill in the art from this disclosure, in one or more embodiments.

It should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

What is claimed is:

1. A system for monitoring pressure trends in an inferior vena cava of a subject, the system comprising:
a pressure measurement device implanted in the inferior vena cava of the subject, the pressure measurement device including a pressure sensor and a transceiver, the pressure measurement device configured to acquire pressure measurements and to wirelessly transmit the acquired pressure measurements; and
a pressure monitor device configured to be worn by the subject externally, the pressure monitor device including a transceiver and system electronics, the pressure monitor device configured to wirelessly receive pressure measurements from the pressure measurement device, to determine a trend in the acquired pressure measurements, and to automatically generate an alert responsive to the determined trend exceeding a predetermined threshold.

2. The system of claim 1 wherein the predetermined threshold is an increase of at least 50% within a period of 24 hours.

3. The system of claim 1 wherein the pressure measurement device includes a stent to which the pressure sensor and the transceiver are secured.

4. The system of claim 3 wherein the stent is configured to expand to a diameter of at least 15 mm and less than or equal to 35 mm

5. The system of claim 3 wherein the stent includes material that is configured to change diameter after implantation, wherein the diameter is configured to change between 20 mm and 35 mm

6. The system of claim 3 wherein the stent includes one or more anchors for securing the stent within the inferior vena cava.

7. The system of claim 3 wherein a radial force applied by the stent is sufficient to secure the stent within the inferior vena cava.

8. The system of claim 1 wherein the pressure sensor comprises an encapsulated pressure transducer.

9. The system of claim 1 wherein the transceiver is configured to transmit radio frequency signals to the pressure monitor device.

10. The system of claim 1 wherein the pressure monitor device is implemented in a belt.

11. A kit comprising packaging and the system of claim 1, the packaging configured to secure the pressure measurement device and the pressure monitor device.

12. A method for monitoring pressure in an inferior vena cava of a subject to diagnose early signs of acute decompensated heart failure, the method comprising:
intermittently measuring blood pressure in the inferior vena cava;
determining, using a processor, a trend in blood pressure in the inferior vena cava based on the intermittent blood pressure measurements;
determining, using a processor, that the determined trend satisfies alert criteria wherein the alert criteria includes an increase in pressure of at least 50% within a 24-hour window; and
indicating a diagnosis of fluid overload responsive to the determination that the determined trend satisfies the alert criteria.

13. The method of claim 12 wherein intermittently measuring blood pressure comprises acquiring blood pressure measurements using a pressure sensor at least two times in a 24-hour period.

14. The method of claim 12 wherein determining the trend in blood pressure includes calculating a difference in pressure measurements over a pre-determined period of time.

15. The method of claim 14 wherein the pre-determined period of time is 24 hours.

16. The method of claim 12 wherein determining the trend in blood pressure includes calculating a slope of a fit to acquired pressure measurements.

17. The method of claim 12 wherein indicating the diagnosis of fluid overload comprises generating a visual alert.

18. The method of claim 12 wherein indicating the diagnosis of fluid overload comprises generating an audible alert.

19. The method of claim 12 further comprising displaying a recommendation to increase intake of oral diuretics responsive to the determination that the determined trend satisfies the alert criteria.

20. The method of claim 12 further comprising playing an audible message that recommends increasing intake of oral diuretics responsive to the determination that the determined trend satisfies the alert criteria.
